(19) United States
(12) Patent Application Publication
(10) Pub. No.: US 2009/0138064 A1
(43) Pub. Date: May 28, 2009

(54) Garment for Providing Back Support and Thermal Therapy

(76) Inventor: Daya Ananda Horn, Williamson, NY (US)

Correspondence Address: LAW OFFICE OF TRACY P. JONG, 2775 BUFFALO ROAD, SUITE 3, ROCHESTER, NY, 14624, US

(21) Appl. No.: 11/945,609

(22) Filed: Nov. 27, 2007

(51) Int. Cl. A61F 7/00; A41C 3/00; A61F 5/00

(52) U.S. Cl. 607/108; 450/2; 602/19; 607/112; 607/96

(57) ABSTRACT

A garment combines features of posture braces, compression garments and thermal therapy devices in a single garment constructed in one piece and folded and reversibly affixed to form an undershirt-like structure fitting around the torso under the arms, thereby covering the back and extending between the cervical spine and the lumbo-sacral spinal region. On the back and front of the garment are a plurality of pouches which can contain a thermal unit which can heat or cool the back. In this manner, the garment can apply variable compression to the back and/or torso and/or abdomen horizontally and/or vertically, and can, independently of the compression, also, apply heat or cold to the back and/or torso.

### BACKGROUND OF THE INVENTION

1. Field of the Invention

This invention relates to garments for the back and/or torso, and in particular, to garments for therapeutic compression and temperature treatment for the back and/or torso.

2. Background of the Invention

There are numerous medical conditions which result in pain and discomfort to the back. Certain medical procedures may also result in back pain. Various chemical agents such as narcotic analgesic and anti-inflammatory agents may be used to relieve the pain and discomfort but these therapeutic agents often have undesirable side effects, may not be tolerated by the patient, or may have inadequate efficiency. A preferred treatment regimen often involves thermal treatment to certain areas of the back, particularly cold therapy for reduction in local inflammation. Another treatment involves support garments that provide support for the bones and muscles. Yet another treatment involves compression of the tissues to reduce inflammation.

Some of the medical conditions which lead to pain and discomfort in the back include, for example, traumatic injury, lumbar sprains, thoracic sprains, post-surgical disk injuries, arthritis, SI joint discomfort, sciatica, piriformis syndrome, general back muscle soreness, back spasms, back stiffness, and the like.

A treating health care professional may request the patient apply thermal therapy to a portion of the body as part of a desired medical treatment. Thus, cold may be applied to reduce swelling or heat may be applied to assist in ameliorating the symptoms or conditions of other maladies.

The prior art discloses U.S. Pat. No. 6,464,717, US20010037076A1, U.S. Pat. No. 6,440,159, U.S. Pat. No. 4,805,620, U.S. Pat. No. 4,976,262 and US20070249264A1. These prior art patented devices do not cover the majority of the torso including, for example, ribs, diaphragm, abdomen, and hips and the back from the cervical spine to the lumbo-sacral spinal region.

The prior art discloses various lumbar wraps, however, these devices are limited to providing thermal treatment and/or support to the lumbar region. The prior art also discloses various compression garments, but they do not simultaneously provide for thermal therapy.

Other prior art products marketed include “The Active Wrap Back Hot/Cold Therapy System sold by Active Wrap, Inc. and the “Lastic Lumbar Support with Insert” sold by Hely & Weber. These prior art patented devices do not cover the majority of the torso including, for example, ribs, diaphragm, abdomen, and hips and the back from the cervical spine to the lumbo-sacral spinal region. The Active Wrap Back Hot/Cold Therapy System provides a single 7×10 ice pack inserted into a wide belt-like structure providing light compression. This does not target certain areas of the back nor provide skeletal support features. The Lastic Lumbar Support with Insert does not provide thermal therapy and its compression and/or support are limited to the lumbar region.

Two prior art devices marketed under the trade names “Kool Max Cold Pack Body Cooling Vest” sold by Polar Products, Inc., 540 South Main Street, Akron, Ohio 44311 www.polarsoftice.com and “Mobil Ice Reusable Cold & Hot Compression Pack” and “Mobile Ice Vest” sold by Grucox Medical Sports Products, www.grucox.com provide for torso coverage, however, the Mobile Ice Vest is designed to decrease the core body temperature and not to address spinal pain and skeletal support needs. The Mobile Ice Compression pack is designed for treatment of sports injuries and also not for spinal pain and skeletal support needs.

These prior art devices also do not have true male and female versions, only unisex versions. More specifically, these prior art devices are not designed to address the needs of pregnant or postpartum women. Maternity specific products in the prior art include a device sold under the trade name “Prenatal Cradle,” various maternity belts and the devices disclosed in U.S. Pat. No. 4,822,317 and U.S. Pat. No. 4,952,192. These devices do not provide for a nursing bra, full torso coverage, spinal support, or have pouches for hot/cold therapy. Additionally, none of these devices address the postpartum period and nursing.

Devices for applying thermal therapy to a body portion such as a joint or muscle portion are known. For example, U.S. Pat. No. 4,964,402 (Grim, et al.) discloses a device in which a gel pack is positioned against a body part. The gel pack may be heated or cooled so that the desired therapy is applied as desired.

U.S. Pat. No. 4,972,832 (Trapini) discloses another device for strapping a thermal therapy unit to a body portion. U.S. Pat. No. 4,976,262 (Palmacci) discloses a device for attaching thermal therapy devices to a body portion in which VELCRO® type connectors are used to connect ends of a wrapping or securing structure. U.S. Pat. No. 5,148,804 (Hill, et al.) shows a device with a pocket for a thermal pack.

Therapy wraps are shown with structure for attachment to a joint like a knee and along a body limb such as the leg or arm. U.S. Pat. No. 4,964,402 (Grim, et al.) shows use around the ankle, knee, and wrist. U.S. Pat. No. 4,976,262 (Palmacci) shows several different structures to hold a thermal device like an ice pack to limb joints and also a structure to hold a thermal device to the shoulder using elastic or stretchable material.

None of the prior art devices are meant for, or appropriate for use under a post-operative brace (such as a TLSO or LSO).

The disclosed devices in the art do not combine skeletal support, compression therapy and thermal therapy. Additionally, the prior art devices are designed and intended for use after an injury, not as a daily undergarment with support and preventative therapeutic benefits. The prior art devices are not designed for freedom of movement by a user and displace when the arms are raised. A single device in which support, compression and thermal therapy may be applied to precise locations on the back is desired and not known.

Thus it is desirable to provide an improved garment that improves upon the prior art and eliminates one or more of its shortcomings and limitations.

The present invention overcomes these problems and limitations by providing a treatment garment which applies compression and thermal treatment variably and independently to the back and torso. In addition, the present invention provides for compression both horizontally and vertically across the back and torso.

### SUMMARY OF THE INVENTION

The garment of the present invention wraps around the back and upper body in an annular fashion, extending between the cervical spine and the lumbo-sacral spinal region. The garment has a front side and back side, an upper edge and a bottom edge. Upper edge has a pair of shoulder straps oriented towards the back side of the garment (back shoulder straps) and a pair of shoulder straps oriented towards the front side of the garment (front shoulder straps). Each the front and back of the garment has two hook or pile fasteners on the peripheral surface. Disposed on the back side is a hook or pile fastener on a first end while the front side has a hook or pile fastener on the second end of the garment. The mating hook or pile fasteners are located in the center portion of each the front and back sides, located such that they are disposed over the rear hip area when the garment is worn by a user. The front shoulder straps have two mating hook or pile fasteners on the outside surface. The back shoulder straps have a loop configured to receive the corresponding front shoulder straps which then fold down upon themselves such that the mating hook and pile fasteners are in contacting engagement with one another.

On the back of the garment are a plurality of pouches which can contain a thermal unit such as, for example, a gel-pack, which can heat or cool the back. In this manner, the garment of the present invention can apply variable compression to the back and/or torso horizontally and/or vertically, and can, independently of the compression, also, apply heat or cold to the back and/or torso.

The garment of the present invention combines features of posture braces, compression garments and thermal therapy devices in a single garment in male and/or female embodiments that permit freedom of movement and comfort for the user. The garment of the present invention may be constructed in one piece and folded and reversibly affixed to form an undershirt-like structure fitting around the torso under the arms, thereby covering the back.

It is an object of the present invention to provide a garment can be constructed of a single piece of material.

It is a further object of the present invention to provide a garment that can simultaneously apply horizontal and vertical compression to the back and/or torso variably and independently.

It is a further object of the present invention to provide a garment that can apply thermal treatment to the back and/or torso independent of the degree or nature of compression produced by the garment.

It is a further object of the present invention to provide a garment that can provide compression and/or thermal treatment simultaneously or independently to relieve pain and/or discomfort of the back.

It is yet another object of the present invention to provide a garment that is durable and reliable.

It is yet another object of this invention to provide a garment that is economical from the viewpoint of the manufacturer and consumer, are susceptible of low manufacturing costs with regard to labor and materials, and which accordingly are then susceptible of low prices for the consuming public, thereby making it economically available to the buying public.

Whereas there may be many embodiments of the present invention, each embodiment may meet one or more of the foregoing recited objects in any combination. It is not intended that each embodiment will necessarily meet each objective.

Thus, having broadly outlined the more important features of the present invention in order that the detailed description thereof may be better understood, and that the present contribution to the art may be better appreciated, there are, of course, additional features of the present invention that will be described herein and will form a part of the claimed subject matter.

In this respect, before explaining at least one embodiment of the invention in detail, it is to be understood that the invention is not limited in its application to the details of construction and the arrangements of the components set forth in the following description or illustrated in the drawings. The present invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the invention be regarded as including such equivalent construction insofar as they do not depart from the spirit and scope of the conception regarded as the present invention.

### BRIEF DESCRIPTION OF THE DRAWINGS

The invention will be described by reference to the specification and the drawings, in which like numerals refer to like elements, and wherein:

FIG. 1 is a plan view of a back side of a male embodiment of a garment;

FIG. 2 is an orthogonal view of a human and a spine;

FIG. 3A is a perspective view of a pouch;

FIG. 3B is a sectional view of a pouch;

FIG. 4 is a plan view of a front side of a female embodiment of the garment;

FIG. 5 is a plan view of a back side of a female embodiment of the garment depicted in FIG. 4;

FIG. 6A is a back orthogonal view of an auxiliary support strap;

FIG. 6B is a front orthogonal view of an auxiliary support strap;

FIG. 6C is a back orthogonal view of a thermal support strap;

FIG. 6D is a front orthogonal view of a thermal support strap;

FIG. 7 is a perspective view of a garment depicted in FIG. 1;

FIG. 8A is a left orthogonal view of a female embodiment of the garment depicted in FIG. 4;

FIG. 8B is a right orthogonal view of a female embodiment of the garment depicted in FIG. 4;

FIG. 9 is a perspective view of garment depicted in FIG. 1 and an auxiliary support strap depicted in FIG. 6A worn by a human;

FIG. 10 is a left orthogonal view of a male embodiment of the garment depicted in FIG. 1 worn by a human; and

FIG. 11 is a front orthogonal view of a bra portion.

The drawings are not to scale, in fact, some aspects have been emphasized for a better illustration and understanding of the written description.

### DETAILED DESCRIPTION OF A PREFERRED EMBODIMENT

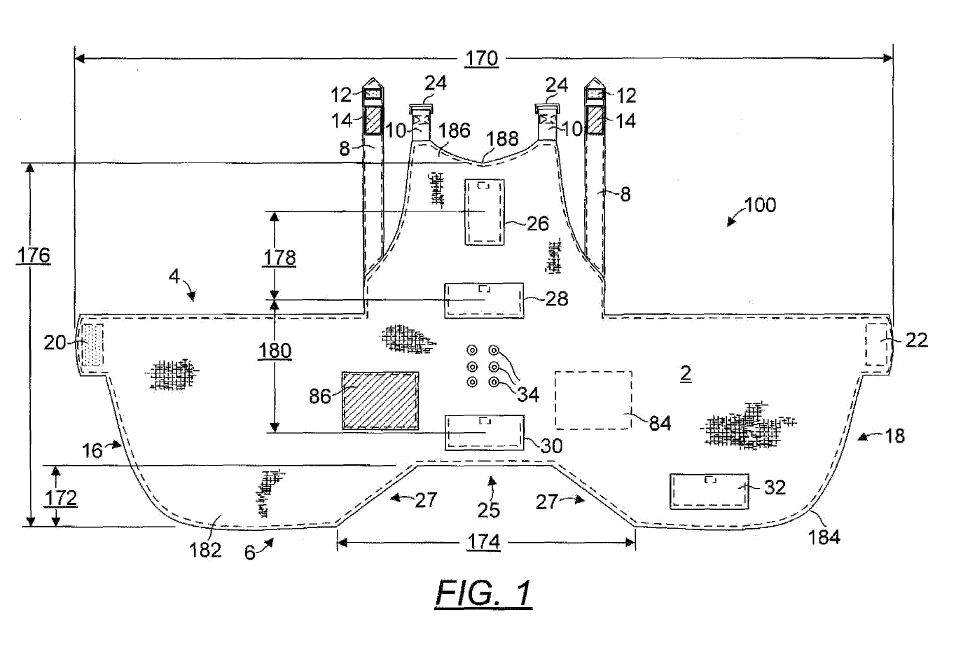

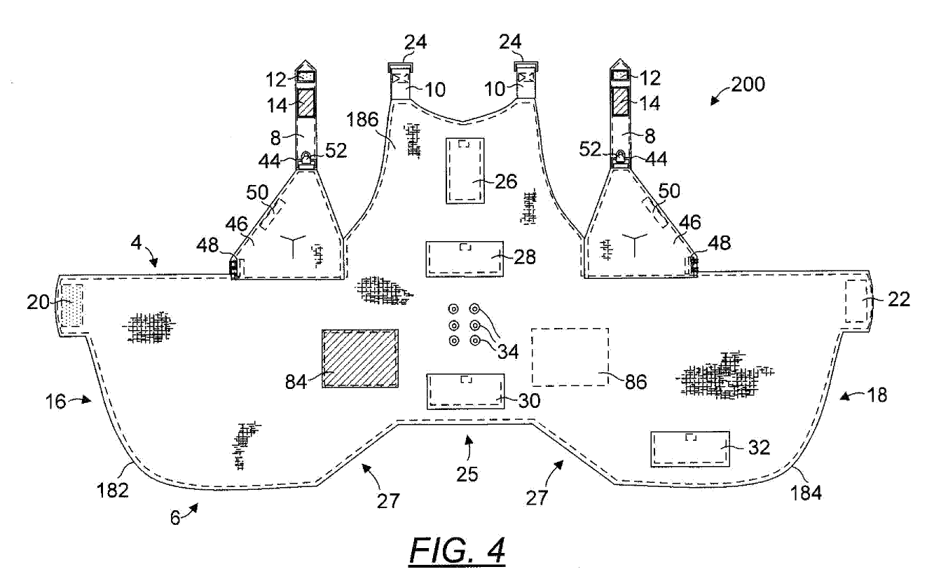

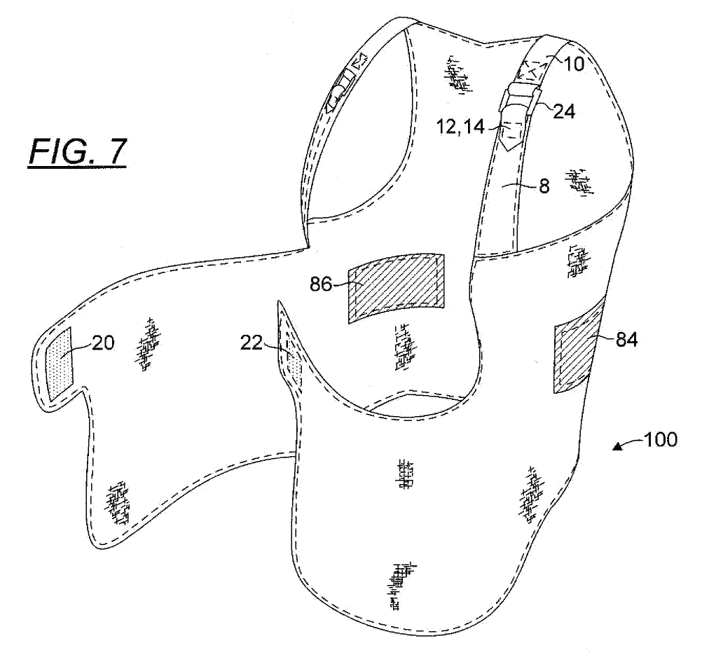

FIG. 1 is a plan view of a back side of a male embodiment 100 of a garment. The garment 100 is sized for positioning about the torso. It is worn in an annular manner about the torso and is disposed about at least a portion of the thoracic spine and at least a portion of the lumbar spine when worn by the user. The garment 100 of the present invention combines features of posture braces, compression garments and thermal therapy devices in a single garment that permits freedom of movement and comfort for the user. The garment 100 of the present invention may be constructed in one piece as shown in FIGS. 1 and 4 (depicted and described in detail below), and folded and reversibly affixed to form an undershirt-like structure fitting around the torso under the arms, thereby covering the back. Reference is made to FIGS. 7-10 (depicted and described in detail below) for clarification and illustration.

FIG. 1 shows the outside surface of garment 100 of the present invention in an open flat position. The garment 100 has a front side (not visible in this view) and back side 2, an upper edge 4 and a bottom edge 6. Upper edge 4 has a pair of shoulder straps oriented towards the back side of the garment (back shoulder straps) 8 and a pair of shoulder straps oriented towards the front side of the garment (front shoulder straps) 10.

Each the front and back 2 of the garment 100 has two hook or pile fasteners 20, 22, 84, 86 on the peripheral surface. Disposed on the back side 2 is a hook or pile fastener 20 on a first end 16 while the front side has a hook or pile fastener 22 on the second end 18 of the garment 100. The length 170 from first end 16 to second end 18 is from about 25 to about 50 inches (about 63.5 to about 127 centimeters). The mating hook or pile fasteners 84, 86 are located in the center portion 186 of each the front and back 2 sides, located such that they are disposed over the rear hip area when the garment is worn by a user. (The location of the hook or pile fasteners 22, 84 on the front side that are not visible in this view in FIG. 1 may be discerned from the stitching outline visible on the back side 2 at 22, 84.)

The front shoulder straps 8 have two mating hook or pile fasteners 12, 14 on the outside surface. The back shoulder straps 10 have a loop 24 configured to receive the corresponding front shoulder straps 8 which then fold down upon themselves such that the mating hook and pile fasteners 12, 14 are in contacting engagement with one another.

The hook or pile arrangements 12, 14 shown in the various Figures may have either a hook fastener or a pile fastener, but the element to which each locks will have the opposite fastener. The hook and pile fastener is similar to that provided under the registered trademark VELCRO®. Although hook and pile fasteners are preferred, for each hook or pile fastener on the garment 100, 200, other reversible closing or locking mechanisms may be used, such as, for example, snaps, belts, ties, hooks, clips and the like.

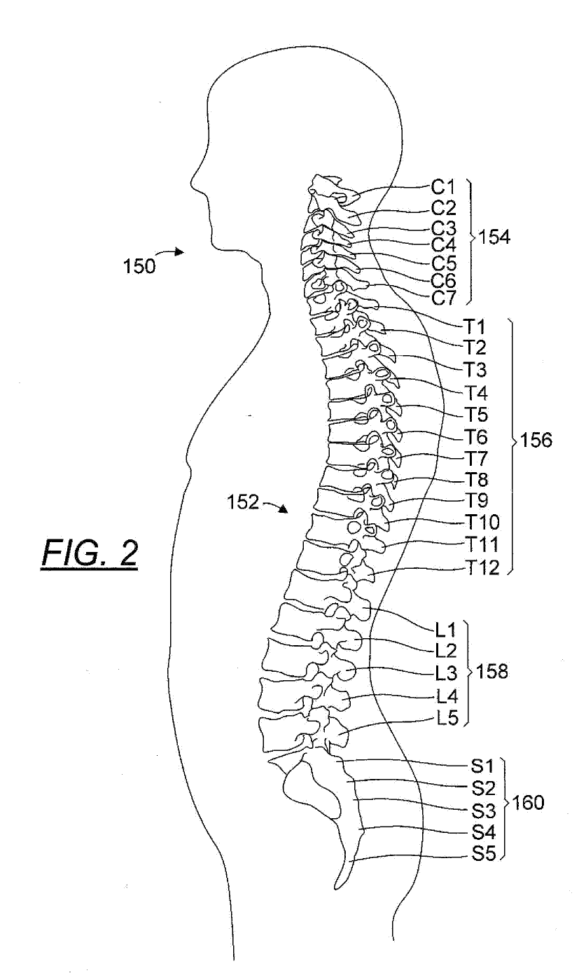

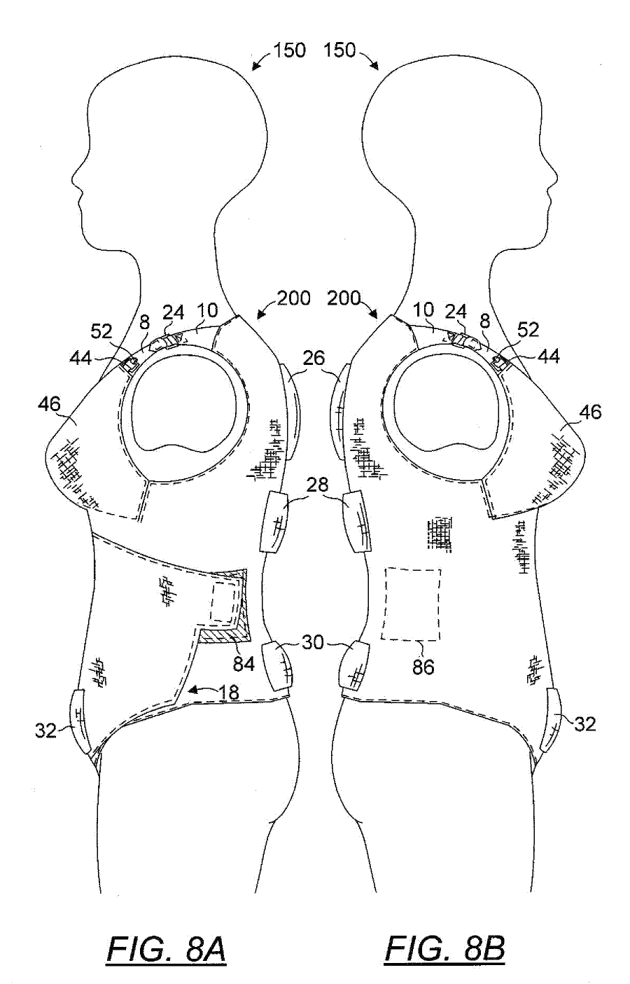

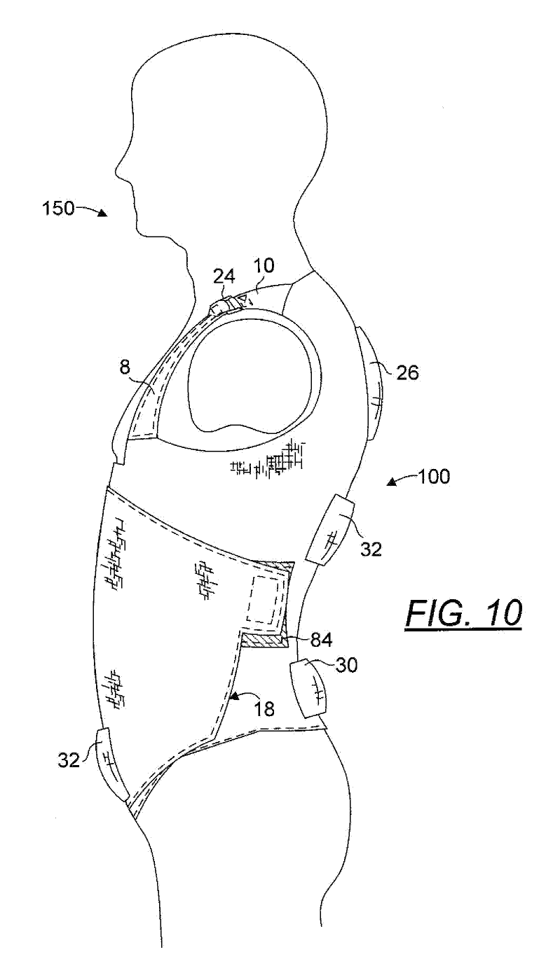

FIG. 2 depicts a human 150 and his spine 152. FIG. 8A depicts a female wearing the female version of the garment 200 and FIG. 10 depicts a male wearing the male version 100 of the garment. Referring to FIGS. 2, 8A and 10, an important feature of the garment 100 is that it extends from the thoracic spinal region 156 to the sacral spinal region 160. It 100 extends from the lower neck region to the pelvic area. Preferably, the top 188 of the back side 2 is disposed over C7 when the garment 100 is worn by a user. In one embodiment, the distance between the top of the back side 2 and the lower edge 6 is from about 18 inches to about 24 inches.

Referring to FIGS. 1 and 2, in one embodiment, the garment 100 covers at least a portion of the cervical spine 154 (at least a portion of C1-C7), the entire thoracic spine 156 (all of T1-T12), the entire lumbar spine 158 (all of L1-L5), and at least a portion of the sacral spine 160 (at least a portion of S1-S5). In another embodiment, the garment covers the back between T1 and L3. In one embodiment, the garment covers the back between C7 and L4. In one embodiment, the garment covers the back between T2 and L5.

Referring to FIG. 2, the adult spine 152 is made up of 24 vertebrae stacked on top of each other from the bottom of the skull to the pelvis. The sacrum is the triangular bone just below the lumbar vertebrae 158. The sacrum 160 has five segments or sacral vertebrae (S1-S5) fused together into one large bone. This triangular bone fits between the two halves of the pelvis, connecting the spine to the lower half of the body. The spine 152 above the sacrum 160 consists of the cervical spine 154 (seven bones or cervical vertebrae in the neck given the labels C1-C7), the thoracic spine 156 (twelve bones or thoracic vertebrae in the chest given the labels T1-T12) and the lumbar spine (five bones or lumbar vertebrae in the lower back given the labels L1-L5). The cervical spine 154 supports the head and connects to the thoracic spine 156. The seventh cervical segment C7 functions as a transitional segment from the neck to the thoracic spine 156. The rib cage of the chest is attached to the thoracic spine 156 at each level. The lumbar spine 158 is the lower back and is made up of five or six vertebrae, depending on the individual.

Referring again to FIGS. 1 and 2, one embodiment of garment 100 comprises at least two pouches configured to receive a thermal unit for delivering thermal therapy to a portion of the torso. Preferably, these pouches include at least two the following pouches depicted in FIG. 1: an upper thoracic pouch, a thoracic pouch, a lumbar pouch, an abdominal pouch and a breast pouch. The upper thoracic pouch delivers thermal therapy to at least a portion the cervical spine and at least a portion of the thoracic spine. The upper thoracic pouch is disposed about at least two vertebrae in the region of T1, T2, T3, T4, T5, T6 and T7. The thoracic pouch delivers thermal therapy to at least a portion the thoracic spine and is disposed about at least two vertebrae in the region of T9, T10, T11 and T12. The lumbar pouch delivers thermal therapy to at least a portion of the lumbar spine and at least a portion of the sacral spine. The lumbar pouch is disposed about at least two vertebrae in the region of L3, L5, L5, S1 and S2.

One advantage of the garment 100 of the present invention is that most users can put it on and secure it without assistance. The closures 12, 14, 84, 86 and adjusting mechanisms are disposed on the front and sides and are easy to use. Stretching and straining is minimized or alleviated. Thus, a patient can use the garment 100 during recuperation and return to activities of daily living without assistance. A user may also wear the garment 100 on a daily basis as an undergarment in the same manner as an undershirt or a bra.

Referring again to FIG. 1, the garment 100 may be constructed to be closed and locked on either the right side of the torso or on the left side by changing the orientation of the first end 16 and the second end 18 of the garment 100, with corresponding changes in the orientation of the hook and pile fasteners, as desired. Reference may be had to FIGS. 7-10 for illustration of the garment in use.

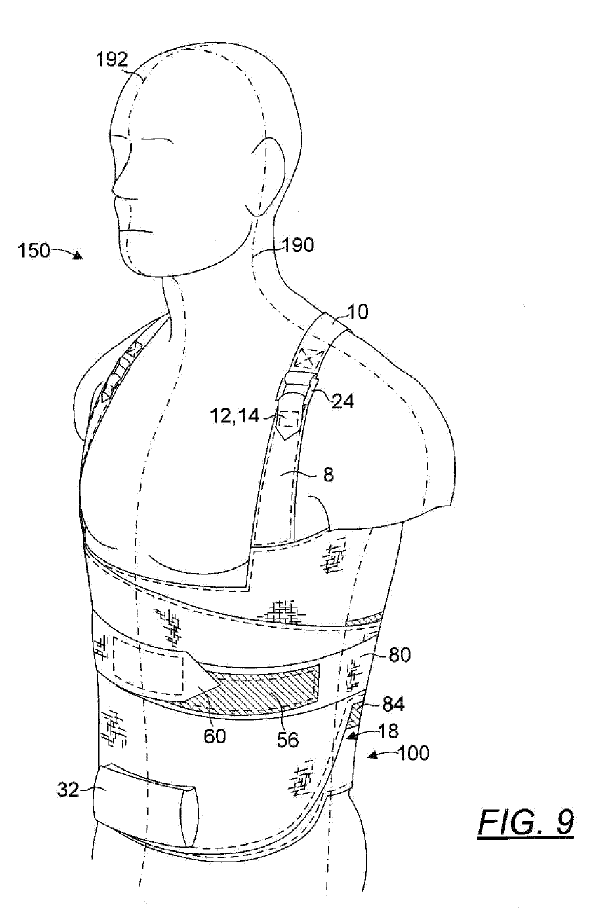

The hook or pile fasteners 84, 86 are located on each the front and back 2 sides of garment 100 in such a manner as they are disposed over the area of the torso that about the rear hip and just behind the arms. As used in this specification, rear hip shall mean the area of the torso that is between the lateral plane 190 and the midline planes 192 (see FIG. 9) and corresponding to the lumbar spinal region between L3 and L5. In one aspect, hook or pile fasteners 84, 86 are disposed over the area of the torso below the 12^{th} rib and above the gluteus medius and illiac crest when the garment is worn by a user. In one aspect, hook or pile fasteners 84, 86 are disposed over the area of the torso corresponding to the lumbar triangle.

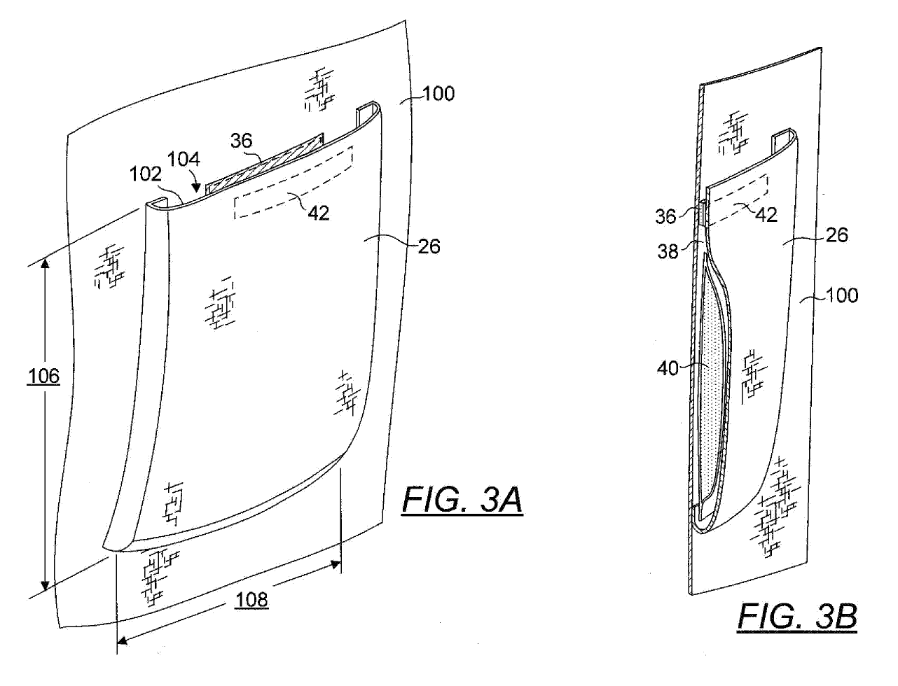

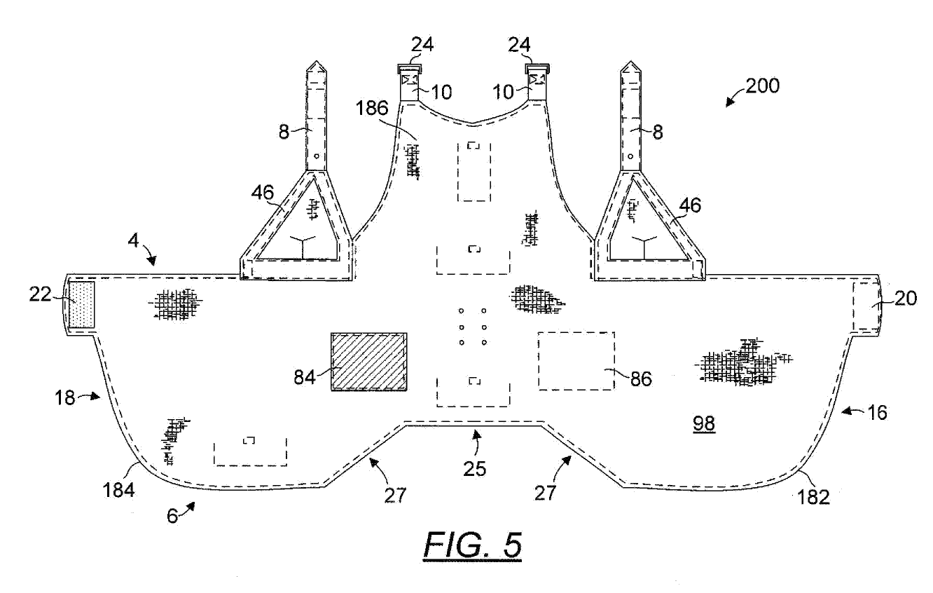

FIG. 3A is a perspective view of a pouch and FIG. 3B is a sectional view of a pouch. FIG. 4 is a plan view of a front side of a female embodiment of the garment. FIG. 5 is a plan view of a back side of a female embodiment of the garment depicted in FIG. 4.

FIG. 5 depicts the front side 98 of a female version 200 of the garment. A hook or pile fastener 22 is disposed on the periphery of the second end 18 of the garment 200 and may be from about one square inch to about 36 square inches (about 232.3 square centimeters) in size. The mating hook or pile fastener 86 is disposed on the exterior peripheral surface of the back side 2 of the garment 100 in the central portion 186. A similar corresponding set of hook or pile fasteners 20, 84 are disposed on the first end 16 of the back side 2 and the central portion 186 of the front side 98. Preferably, these hook and pile fasteners 84, 86 are disposed on the portion of the garment 200 that is over the rear hip. In a preferred embodiment, these hook or pile fasteners 84, 86 are disposed just to the right and left, respectively, of the row of pouches 26, 28, 30 and between pouches 28 and 30. These hook and pile fasteners 84, 86 may be, but are not required to be larger than the mating elements 20, 22 on the first 16 and second 18 ends of the garment 200. In one embodiment, these hook and pile fasteners 84, 86 are from about one square inch to about 144 square inches (about 929 square centimeters). In a preferred embodiment, the hook or loop fasteners are about 2.5 inches (about 6.35 centimeters) by about 4 inches (about 10.16 centimeters). The larger size of these hook and pile fasteners 84, 86 allows more flexibility in customizing the fit of the garment 200 to bodies of various sizes and shapes. It also provides a more secure connection when the user is moving. As will be apparent, these described elements will also be disposed on the front side of the male version 100 of the garment depicted in FIG. 1.

The garment 100 of the present invention can be used to apply variable compression to the back and/or torso in horizontal and/or vertical directions while, simultaneously and independently, applying heat or cold to the back and/or torso and/or abdomen. The application of compression and heat or cold to the back and/or torso and/or abdomen is useful in relieving pain and discomfort in a variety of conditions and disorders, including, for example, back surgery, general back pain, thoracic surgery and any like conditions which produce trauma or induce pain in the back and/or torso.

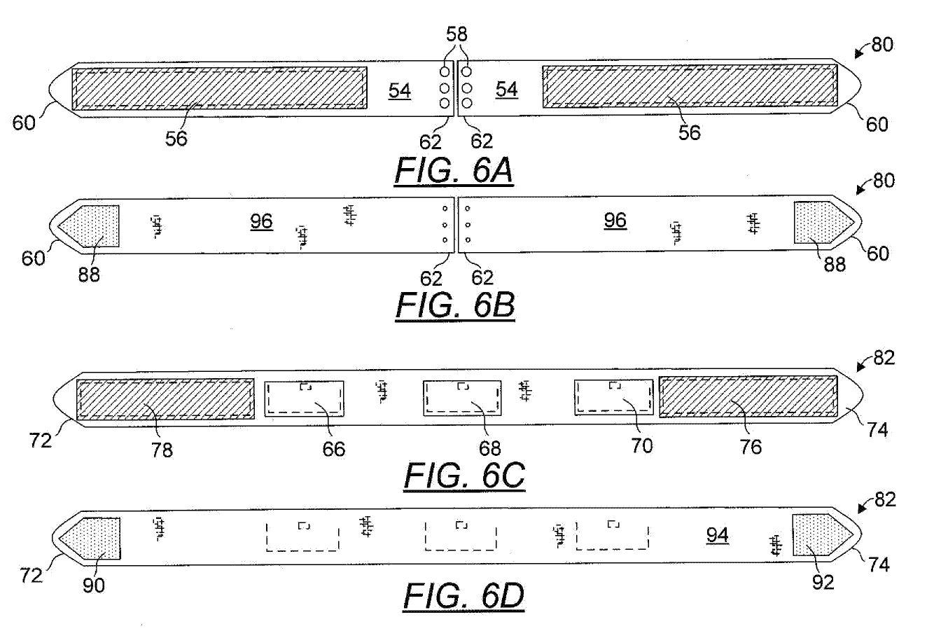

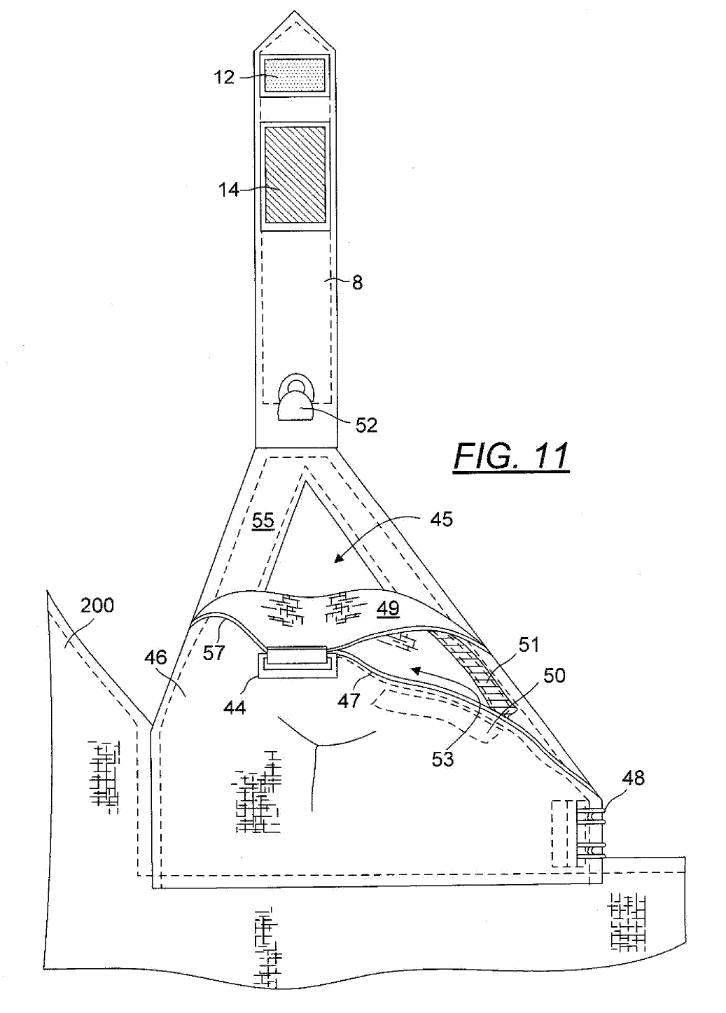

FIG. 6A is a back orthogonal view of an auxiliary support strap and FIG. 6B is a front orthogonal view of an auxiliary support strap. FIG. 6C is a back orthogonal view of a thermal support strap and FIG. 6D is a front orthogonal view of a thermal support strap. FIG. 7 is a perspective view of a garment depicted in FIG. 1. FIG. 8A is a left orthogonal view of a female embodiment of the garment depicted in FIG. 4. FIG. 8B is a right orthogonal view of a female embodiment of the garment depicted in FIG. 4. FIG. 9 is a perspective view of garment depicted in FIG. 1 and an auxiliary support strap depicted in FIG. 6A worn by a human. FIG. 10 is a left orthogonal view of a male embodiment of the garment depicted in FIG. 1 worn by a human. FIG. 11 is a front orthogonal view of a bra portion.

Referring to FIGS. 2 and 7-10, the garment 100 in use is wrapped around the upper torso in an annular fashion under the arms and extends from the cervical spinal region 154 to the sacral spinal region 160. A first end 16 and/or a second end 18 of the garment 100 is reversibly attached to the hook and pile fasteners 84, 86 applying any degree of compression desired prior to attaching and reversibly locking each end 16, 18 of the garment 100 to the hook and pile fasteners 84, 86. Compression is, thus, applied horizontally across the back and/or torso and/or abdomen from the bottom portion to the top portion of the torso and from the lower cervical spinal region (C5-C7) 154 and/or upper thoracic spinal region (T1-T3) to the upper sacral spinal region (S1-S2) 160 and/or lower lumbar spinal region (L3-L5) 158 on the back.

The back shoulder straps 10 are then pulled over the shoulders towards the front shoulder straps 8 and the front shoulder straps 8 pass through the loop 24, folding back over onto itself and reversibly connecting the corresponding hook and pile fasteners 12, 14. By pulling the front shoulder straps 8 tighter, any degree of compression can be created. Compression is, thus, applied vertically across the back and/or torso and/or abdomen from one side of the back and/or torso and/or abdomen to the other.

The horizontal and vertical compressions can be adjusted variably and independently via the hook or pile fasteners (e.g. 84, 86, 12, and 14). Compression therapy is beneficial to alleviate or prevent inflammation. It can also reduce bleeding at post-operative incisions and surgery sites.

FIG. 3A depicts a pouch 26 and FIG. 3B depicts a pouch 26 containing a thermal unit 40. Once the desired compression is created, a thermal unit 40 can be placed against the back and/or torso and/or abdomen to provide heat or cold treatment and be held in place in one of more of the pouches 26, 28, 30, 32. The thermal unit 40 can be applied or removed independently of the application of compression and without altering or interfering with the compression generated by the garment 100 or shoulder straps 8, 10.

Referring to FIGS. 1 and 2 (and correspondingly to FIG. 4), in a preferred embodiment, three pouches 26, 28, 30 are disposed on the back side 2 of the garment 100, one each on the portion of the garment 100 that is located over the upper thoracic region, the thoracic spinal region and the lumbar spinal region 158, 160 (below the natural sway of the lumbar spine). In one embodiment, an upper thoracic pouch 126 is disposed over at least a portion of the region between C7 and T7. Preferably, thermal therapy in this upper thoracic pouch 126 radiates to at least a portion of the cervical spine 154 and the thoracic spine 156.

In one embodiment, a thoracic pouch 128 is disposed over at least a portion of the region between T9 and T12. Thermal therapy in this thoracic pouch 128 radiates to at least a portion of the thoracic spine 156. In one embodiment, a lumbar pouch 130 is disposed over at least a portion of the region between L3 and S2. Preferably, thermal therapy in this lumbar pouch 130 radiates to at least a portion of the lumbar spine and sacral spine.

In a preferred embodiment, the distance 178 between the centerpoint of upper thoracic pouch 126 and the centerpoint of thoracic pouch 128 is from about 3 to about 10 inches (about 7.62 to about 25.4 centimeters). In a preferred embodiment, the distance 180 between the centerpoint of thoracic pouch 128 and the centerpoint of lumbar pouch 130 is from about 4 to about 15 inches (about 10.16 to about 38.1 centimeters).

There is no known prior art device that provides thermal therapy at precisely located positions along the spine 152 or that provides for thermal therapy at multiple spinal locations simultaneously. The garment 100 of the present invention provides for thermal therapy at precisely located positions along the spine 152. It also provides for thermal therapy at multiple spinal locations simultaneously. The garment 100 of the present invention allows thermal therapy to be precisely directed at various spinal portions of the back while a user simultaneously bends turns, moves, walks, sits and the like. No other known device allows for this flexibility and precision simultaneously.

In one embodiment, there is also a pouch 32 disposed on the portion of the garment 100 that is located over the supra pubic and/or abdominal region, the abdominal pouch 32. The abdominal pouch 32 may be disposed along either the back side 2 or front side (e.g. 98 on the female version in FIG. 4) peripheral surface as desired. The abdominal pouch 32 may be advantageously used for treatment of lower abdominal or supra pubic areas, for example, following abdominoplasty, tummy tucks, caesarian deliveries, hysterectomy, hernia repair procedures, endoscopic abdominal procedures, and the like.

The upper thoracic pouch 26, the thoracic pouch 28, and the lumbar pouch 30 may be disposed along either the back side 2 or front side (e.g. 98 on the female version in FIG. 4) peripheral surface as desired. Disposing the pouches 26, 28, 30, 32 on the front side (e.g. 98 on the female version in FIG. 4) peripheral surface may provide advantages such as greater thermal therapeutic effects and a smoother contour or profile of the garment 100 during wear under regular clothing. Disposing the pouches 26, 28, 30, 32 on the exterior peripheral surface of the back side 2 may provide advantages such as easy accessibility for insertion and removal of the thermal units (see 40 of FIG. 3B).

Referring again to FIG. 2, the normal spine 152 has an S-shaped curve when viewed from the side. This shape allows for an even distribution of weight and flexibility of movement. The spine 152 curves in several ways. The cervical spine 154 curves slightly inward, sometimes described as a backward C-shape or lordotic curve. The thoracic spine 156 curves outward, forming a regular C-shape with the opening at the front or a kyphotic curve. The lumbar spine 158 curves inward and, like the cervical spine 154, has a lordotic or backward C-shape. In a preferred embodiment, the upper thoracic pouch 26, the thoracic pouch 28, and the lumbar pouch 30 are located on the back 2 of the garment 100 to correspond to each of these natural spinal curves where pain is most often experienced.

Preferably, the upper thoracic pouch 26 is located between the shoulder blades to provide the maximum freedom of motion. In one aspect, the upper thoracic pouch 26 is located over the first curve of the spine. In the embodiments depicted in FIGS. 1 and 4, the thoracic pouch 28, the lumbar pouch 30 and the abdominal pouch 32 are horizontally disposed and the upper thoracic pouch 26 is vertically disposed.

Referring to FIGS. 3A and 3B, a pouch 26 (as well as 28, 30, 32) comprises a pouch wall 102 and an opening 104 which opens into the pouch interior 38. The pouch interior 38 is configured to securely hold a thermal unit 40 for thermal therapy to the human back and/or torso and/or abdomen. The interior of the pouch 38 is preferably has a length 106 and width 108 of from about 5.5 inches (about 13.97 centimeters) to about 14 inches (about 35.56 centimeters). In a preferred embodiment, each pouch 26, 28, 30, 32 has a dimension 106, 108 of from about six inches by about nine inches. When the thermal unit 40 is disposed in the pouches 26, 28, 30, 32, the thermal effect is radiated to nearby areas. Thus, preferably, the upper thoracic pouch radiates thermal therapy to the lower cervical region and the lumbar pouch radiates thermal therapy to the sacral region.

The opening 104 is selectively sealable by means of mating hook and loop portions 36 (the corresponding portion is discernible from the stitching outline 42 visible along the exterior peripheral surface of the pouch wall 102. By way of illustration, such hook and loop portions 36 may be VELCRO® strips. As will be apparent to one skilled in the art, any suitable means can seal the opening 104 of the pouch, including a zipper, male and female connectors, snaps, ties, clips, and any other type of suitable fastener. Alternatively, the pouch 26, 28, 30, 32 can be made sufficiently elastic and taut to ensure that the opening 104 has to be pulled open to allow a thermal unit 40.

Thermal therapy may include the application of heat and/or cold to a treatment area. The thermal unit 40 may take many forms such as a cold pack, a hot pack, or a dual hot-cold pack. The dimensions 106, 108 of the pouch 26, 28, 30, 32 can be made to fit a range of cold or hot packs or a specifically sized cold or hot pack. Common thermal packs are available in small sizes of about 5×8 inches (20.32 centimeters) to large sizes of about 11×14 inches (35.56 centimeters).

As will be apparent, thermal units 40 may also be disposed on the front or sides between the right abdominal flap 184 and left abdominal flap 182 of the garment 100 when wrapped across the torso. This provides and advantage in allowing for additional thermal therapy to other portions of the torso without the necessity of prealigned pouches.

Any thermal unit 40 that is adapted to fit over the back and/or torso and/or abdomen can be used in the present invention. A cold pack could be, for example, ice secured inside a ziplock bag or even a bag of frozen vegetables. A cold pack could also comprise commercially available products used in coolers, thermal food packaging (e.g., lunch packs) and medical supply sources (first aid kits and institutional use products). By way of illustration, a cold pack that could be suitably used with the present invention includes the Colpac narrow size (3 inches (7.62 centimeters) by 11 inches (27.94 centimeters)) made by Chattanooga, and the Chattanooga Black Urethane Half Size Colpac Ice Pack (6.5 inches (16.51 centimeters) by 11 inches (27.94 centimeters)).

A hot pack could be, for example, heatable beads secured inside a cotton bag. It could also be a heated wet cloth secured inside a ziplock bag. A hot pack could also comprise commercially available products used for hand warmers, thermal food packaging (e.g., lunch packs) and medical supply sources (first aid kits and institutional use products). By way of illustration, a hot pack that could be suitably used with the present invention includes THERAPRO® single use hot pack.

As used in this specification, the term “dual hot-cold pack” refers to packs, which may or may not be reusable, that can be either heated or cooled prior to use. Dual hot-cold packs include gel packs that can be cooled in a freezer or heated in a conventional domestic microwave machine (or its functional equivalent) or by immersion in hot water. A dual hot-cold pack could comprise one of the many commercially available products. The preferred dual hot-cold pack is a gel-pack well-known in the art for applying heat or cold to the body surface. Gel or reusable fluid materials employable in the gel-packs of the present invention for heat exchange are well known in the art. For example, one reusable, heatable/chillable gel employable in the pouches could be composed of approximately 40% glycerin, 52% distilled water, and 8% starch. The preferred gel-pack is reusable, can be heated in any suitable oven or heating device, including, preferably, a microwave oven, and can be placed in a freezer to cool the gel-pack to 0° C. (32 degree Fahrenheit) or lower without causing the gel to solidify.

By way of illustration, a dual hot-cold pack that may be suitably used with the present invention includes Flexi-Pac™ supplied, for example, by Sportstek Physical Therapy Supplies Pty Ltd, 6 Park Road, Oakleigh, Victoria 3166, Australia. Another suitable dual hot-cold pack is the hot-cold small sectional sack (4 inches (10.16 centimeters) in width, 7 inches (17.78 centimeters) high, 11 inches (27.94 centimeters) in length), which can be purchased from Target Stores nationwide (e.g., Target at 5115 Leesburg Pike, Falls Church, Va. 22041-3207 US), or ordered online from Target Stores (part of Target Corp., 1000 Nicollet Mall, Minneapolis, Minn. 55403 and the Jack FroSt™ hot and cold pack supplied by SHOP.COM (item #1001682) headquartered in Monterey, Calif., USA.

The garment 100 of the present invention may be further adapted so that a thermal unit 40, such as, for example, a gel-pack, may be reversibly attached directly to the exterior peripheral surface of the garment 100 directly over the back, on either the front or back side 2, without the use of pouch for holding the thermal unit 40 against the back. The garment 100 may have a belt, strap or tie to reversibly attach a thermal unit 40 to the garment 100. Alternatively, a hook or pile fastener and the thermal unit 40 may have a corresponding reciprocal hook or pile fastener so that the thermal unit 40 will adhere to the exterior peripheral surface over the back when placed against the back. Although hook and pile fasteners are preferred, other reversible closing or locking mechanisms maybe used, such as, for example, snaps, clips and the like.

In yet another embodiment (not depicted), the garment 100 may be quilted or waffled such that it comprises a plurality of pouches equally spaced throughout the interior and/or exterior peripheral surfaces of the garment 100. Thermal units 40 may be placed in any or all of these pouches as desired by the user. The quilting or waffling may contain uniformly sized pouches or a variety of pouch sizes and/or shapes as desired.

The garment 100 of the present invention may be constructed of any suitable cloth and/or polymeric material. Preferably, the garment 100 is formed of an elastically deformable material and particularly those that are biaxially elastically deformable. The preferred material will be soft, stretchable (in two directions), resilient, and elastic, and be porous or permeable sufficiently to allow moisture to evaporate and escape from the portion of the body covered by the garment 100, and to conduct heat. Several such materials are known in the art and include, for example, those made of polyamide, nylon, polypropylene, and/or polyester fabrics, a closed-cell neoprene, or materials sold under the trade names of SILKYLENE®, TACTEL®, LYCRA® or SPANDEX®. A wetsuit like material may also be used. In one aspect, a blended material with combinations of the foregoing materials are used to form the garment. It is not required that each portion of the garment be comprised of the same material.

In one aspect, a plurality of apertures or holes may be formed through the material to facilitate the transmission of air and moisture therethrough.

In one aspect, the material is treated to have antimicrobial and/or waterproof qualities. Processes and materials for such treatments are well known in the art. These may be especially advantageous in medical settings and postoperative applications. The material is preferably washable and durable. In one aspect, the material is preferably nontoxic, patient safe, latex free and NSI compliant as rash-free.

One of the advantages and applications of the garment 100 is that is can be worn daily under the user's exterior clothing. The garment 100 can be used, for example, as an undergarment (undershirt and/or bra like substitute) to provide general support during the day while bending and standing. If desired, thermal units can be inserted in any of the pouches 26, 28, 30 to alleviate back pain during the course of the day. Similarly, the garment 100 may be worn during recuperation periods to allow a user to return to daily activities of living. The pouches 26, 28, 30, 32 allow a user to apply thermal therapy and compression periodically throughout the course of the day while not being confined to a horizontal position or a chair.

In one embodiment, at least a portion of the garment 100 is formed of a molded fabric along the lower edge 6. In one aspect, the molded fabric may be cut with scissors by a user to create a custom fitting contour for the lower edge 6 of the garment 100 without destroying the integrity of the garment 100. This is especially desirable along the hip and pelvic areas.

Referring again to FIG. 1, one of the important features of the garment 100 of the present invention is the hip cutout 25. One of the drawbacks of other devices is their linear lower edge. During use, the linear lower edge has a tendency of rolling up when a user sits or bends. This distorts the device, creating inconveniences including discomfort, lack of proper compression, dislodging of thermal units, and bulging. The hip cutout 25 of the present invention 100 creates a contour that maximizes freedom of movement while simultaneously preventing “roll up” of the lower edge 6 during movement. This ensures constant and consistent support and compression across the torso, abdomen and back during use. It also retains the proper alignment of the thermal units 40 during use.

Referring again to FIG. 1, the hip cutout 25 is a trapezoid shaped cutout. Distance 172 is from about 2 inches (about 5.08 centimeters) to about 6 inches (about 15.24 centimeters), preferably from about 2.5 to about 4 inches (about 6.35 to about 10.16 centimeters), and distance 174 is from about 16 to about 40 inches in proportion to the size of the garment. The angled portion 27 of the hip cutout 25 is configured such that the lower edge 6 along the hip cutout 25 is disposed generally along the top of the hip and top of the illiac crest during use. The lower edge 6 on either side of the hip cutout 25 is disposed below the pelvic bone during use. The contour may be described as “French cut,” “thigh cut,” or “high cut” in the garment and fashion industry.

The hip cutout 25 also provides a supporting element by aligning more of the body weight over the hips for greater skeletal support. The hip cutout 25 allows the lower edge 6 to reside in the lower pelvic region, thus preventing the weight from being directed below the lower edge 6.

Referring to FIGS. 1, 6A and 6B, a plurality of mechanical fasteners 34 are located in the center portion 186 of the back side 2 on the exterior peripheral surface. Preferably, these are the female portion of a snap and are disposed along the spinal portion of the garment in relative alignment with the three spinal pouches 26, 28, 30. Alternatively, hook and eye components, hook and pile arrangements, zippers, ties, straps and other suitable structures for reversibly and detachably affixing an auxiliary support strap 80 (see FIGS. 6A and 6B) are suitably used. The mating portions 58 of the mechanical fasteners 34 are disposed on the auxiliary support strap 80. While six mechanical fasteners 34 are depicted on the garment 100 in FIG. 1, it is to be understood that any number and configuration may be suitably used to accomplish the intended purpose of these structures.

The auxiliary support straps 80 are depicted in FIGS. 6A and 6B. A thermal support strap 82 is depicted in FIGS. 6C and 6D. They essentially comprise an elastic belt-like structure that may be used to add additional compression, support and thermal therapy to the lower back, especially the lumbar region. Preferably, they 80, 82 are formed of elastically deformable materials described herein with respect to the garment 100.

Additional compression and thermal therapy may also be applied to the lower torso and/or abdominal regions on the front side with thermal support strap 82. The auxiliary support straps 80 and thermal support strap 82 may additionally be wrapped around the torso in any configuration necessary to provide the desired compression or thermal therapy, or to provide increased support to a particular region.

Referring to FIG. 6A depicting the back side 54 of an auxiliary support strap 80, a plurality of mating portions 58 of a mechanical fastener is disposed on a first end 62 of the strap 80. In the embodiment depicted, the mechanical fasteners 58 comprise a male snap. At least a portion of the exterior peripheral surface of the back side 54 is covered with a hook or pile fastener 56. Preferably, at least 50 percent of the exterior peripheral surface of the back side is covered with such hook or pile fastener 56. The greater coverage allows for more flexibility in fitting the auxiliary support strap 80 around bodies of different sizes and shapes.

Referring to FIG. 6B depicting the front side 96 of an auxiliary support strap 80, a hook or pile fastener 88 is disposed on a second end 60 of the strap 80. In one embodiment, hook or pile fastener 88 may be from about one square inch to about 9 square inches (about 58.06 square centimeters).

As will be apparent, the user may use one or both of auxiliary support straps 80 as desired. The thermal support strap 82 may be used in addition to the garment 100, in addition to the auxiliary support straps 80, or even as a stand alone therapeutic device. It is to be understood that no thermal unit pouches may be provided, as well as other numbers of thermal unit pouches as desired, for example, one, two, and four or more.

Referring to FIGS. 1, 6A, 6B and 9, the use of the auxiliary support strap 80 is described. The mechanical fasteners 58 are used to connect the auxiliary support strap 80 to the garment 100 at mechanical fasteners 34 (see FIG. 1). The auxiliary support straps 80 are wrapped around the torso and secured such that the hook or pile fasteners 88 are in contacting engagement with their mating hook or pile fastener 56.

Referring to FIG. 6C depicting the front side of a thermal support strap 82, at least a portion of each end 72, 74 of the exterior peripheral surface of the front side is covered with a hook or pile fastener 76, 78. Preferably, at least 20 percent of the exterior peripheral surface of the back side is covered with such hook or pile fastener 76, 78. The greater coverage allows for more flexibility in fitting the thermal support strap 82 around bodies of different sizes and shapes. Along the remainder of the exterior peripheral surface of the front side are one or more thermal unit pouches 66, 68, 70. The embodiment depicted shows three thermal unit pouches 66, 68, 70, equally spaced along the center portion of the thermal support strap central portion. These pouches 66, 68, 70 are substantially the same as depicted and described with respect to spinal and abdominal pouches 26, 28, 30, 32. As will be apparent, the thermal unit pouches 66, 68, 70 may be located and configured in many ways to accomplish the intended purpose. The thermal support strap 82 may be used to apply additional thermal and compression therapy across the body as desired by the user. The thermal support strap 82 may be wrapped around the torso in any direction desired. In fact, the thermal support strap 82 may be wrapped around other limbs and body contours independently of the garment 100.

Referring to FIG. 6D depicting the front side 94 of a thermal support strap 82, a hook or pile fastener 90, 92 is disposed on each longitudinal end 72, 74 of the strap 82. In one embodiment, hook or pile fastener 90, 92 may be from about one square inch to about 9 square inches (about 58.06 square centimeters).

It is believed that soft tissues around the spine also play a key role in the health of the back. Muscles can cause back pain when the facet joints or other parts of the spine become injured and swell, which can cause the large muscles of the back to contract involuntarily (spasm). Back pain also results from chronic stress causing the muscles to tighten up, using up energy that is needed to hold the spine upright. Tight muscles in the back of the thighs can cause changes in the position of the pelvis, affecting spine movement. The supporting elements described above provide preventative therapeutic effects to address these injuries and pains.

A large, complex group of muscles work together to support the spine and hold the body upright. They also allow the trunk of the body to move, twist and bend in many directions. Three types of back muscles that help spinal function are the extensor muscles, flexor muscles and oblique muscles. Extensor muscles are attached to the back of the spine, and allow us to stand and lift objects. They include the large muscles in the lower back (erector spinae), which help hold up the spine, and gluteal muscles. Flexor muscles are attached to the front of the spine and include the abdominal muscles. They allow one to flex, bend forward, lift and arch the lower back. When the abdominal muscles are weak, the muscles that allow us to bend at the hip get tighter, increasing the curve at the lower back. Oblique muscles are attached to the sides of the spine. They help one to rotate the spine and maintain proper posture.

Approximately half of the ability to bend forward comes from the hips, while the rest comes from the lower back. Because the lower back has a more motion than the thoracic spinel 56 and carries all the weight of the upper body, it is more commonly affected by degenerative disc disease or injuries. The lower back is divided among five motion segments. These are the segments most likely to break down from wear and tear. They are prone to developing conditions, such as osteoarthritis and degenerative disc disease. The point where the lower back joins the sacral region 160 (the L5-S1 joint) can develop pain due to injury. This area receives a great deal of stress and twisting during activities, such as sports or sitting for long periods. The lowest discs (L4-L5 and L5-S1) take a great deal of strain because of their location and the greater weight they support. They more commonly develop herniated, bulging or ruptured disks.

Thermal therapy (hot or cold) can be provided directly to these various spinal regions and muscle groups where pain and distress are commonly experienced.

In one embodiment, the garment provides support with or without the thermal therapy benefits. In this aspect, the thermal unit pouches 26, 28, 30, 32 are optional. In a support garment 100, it is sized for positioning about a torso of a user, has a hip cutout 25, and is disposed about at least a portion of the thoracic spine 156 and at least a portion of the lumbar spine 158 when worn by the user. The support garment 100 is formed from an elastically deformable material and comprises a left abdominal flap 182 and a right abdominal flap 184 that are disposed in overlapping alignment about the abdomen when the garment 100 is worn by the user. It comprises two front shoulder straps 8 and two back shoulder straps 10 that cooperate to apply compression vertically across the torso. The garment 100 has a first connecting section 86 disposed about the left rear hip of the user such that the first connecting section 86 and the right abdominal flap 184 cooperate to apply compression horizontally across the torso. Correspondingly, the garment 100 has a second connecting section 84 disposed about the right rear hip of a user such that the second connecting section 84 and the left abdominal flap 182 cooperate to apply compression horizontally across the torso. The garment may be used in combination with an auxiliary support strap 80 formed from an elastically deformable material and worn on the torso in an annular fashion to apply yet additional compression horizontally across the torso.

FIG. 9 depicts a user wearing the garment 100 and two auxiliary support straps 80 depicted in FIGS. 6A and 6B. On the back side each auxiliary support strap 80 are located the mating mechanical fasteners 58 that allow them to be detachably affixed to the garment 100. In the embodiment depicted, these are male snap portions. As will be apparent, a first end 62 of the auxiliary support strap 80 affixes to the exterior surface of the back 2 of the garment 100. During use, the auxiliary support strap 80 extends around the side of the wearer and the second end 60 is reversibly attached to the front of the user with a hook and pile fastener 56 disposed on the front side 96 of the auxiliary support strap 80. One or two auxiliary support straps 80 may be used as desired. Maximum benefit is realized when two auxiliary support straps 80 are used, one over each side of the back and torso as depicted.

The prior art has flexible, rigid, and semi-rigid spinal orthoses made of everything from metal and fabric to lightweight thermoplastics. They all share in common the objective of delivering an immobilizing three-point force to stabilize weak or injured structures. These include, for example, Thoracolumbar orthotic (TLO), Thoracolumbosacral orthotic (TLSO) also known as (“clamshell,” “Boston braces” and “underarm braces”), Knight-Taylor braces, Jewett hyperextension braces, Chairback braces, lumbosacral orthotic braces (LSO), Williams braces, MacAusland braces, Standard LSO corsets, Rigid LSO braces, and the like.

The garment 100 of the present invention may be advantageously used with these orthoses and their functional equivalents. Many of these prior art orthoses (also known as braces) cause friction and skin breakdown during use. The garment of the present invention is formed of a wearable and comfortable fabric that “breathes” (that is, allows the passage of air and moisture), alleviating friction and skin breakdown during use.

In some postoperative back surgery situations, a patient benefits from thermal therapy and/or compression therapy for relief from pain and/or inflammation. Currently, applying thermal therapy to the back involves the patient lying in a prone or supine position while ice packs are applied to the back. The patient is thus temporarily immobile and otherwise unable to move or orient himself to be more comfortable. Whereas this therapy generally is applied regularly throughout the day in 20 minute increments, this is not optimum since patients are encouraged to get out of bed and increase activity to prevent post operative surgical complications.

Back surgery, as well as other thoracic surgeries, often leaves a patient in considerable post operative pain. Pain relief is available from various analgesics and other pain relievers, however, drug free remedies are desirable. Products that adhere to the skin and change temperature are available in the form of various stick-on wraps and patches. However, these stick-on products are not currently available to provide cold thermal therapy. Even if they were available, they would have drawbacks experienced with the hot patch corollaries in that they have a tendency to roll up on highly contoured areas of the body. Additionally, the adhesives are known to cause skin reactions, skin burns, skin irritations and the like. These single use disposable products also produce an excessive amount of waste and are costly for the user.

Products such as those sold under the trade names BIOFREEZE® and ICY HOT® are available, however these have certain limitations. First, they cannot be self applied to the back in most cases (inability to reach). Secondly, these chemicals can produce undesirable allergic reactions or drug interactions. Third, the disposal of biowaste with these agents can be problematic as they pose a danger to children and pets. Fourth, these ointments and topical preparations have limited duration and often are inadequate relief for neurological and nerve pain.

The garment 100 of the present invention provides a better alternative since it allows a post operative back surgery patient to apply compression and/or thermal therapy under the orthoses, allowing the patient to be out of bed and mobile, walking, in a sitting position, or otherwise more comfortable than in the horizontal face down position. The garment 100 also provides drug-free and chemical free pain relief for these patients. As is known to one skilled in the art, ice packs or cold thermal therapy is one of the few treatments that provide relief to patients experiencing nerve pain and neurological issues. Other medicaments often provide no relief for these genres of pain. The garment 100 provides this thermal therapy specifically directed to various portions of the spine 152, and allows more than one spinal region 154, 156, 158, 160 to receive treatment simultaneously if desired.

In one embodiment, the garment 100 may be worn in combination with an orthotic (not depicted) for the torso. In this embodiment, the garment 100 is sized for positioning about a torso and is disposed about at least a portion of the thoracic spine 156 and at least a portion of the lumbar spine 158 when worn by the user. It is formed from an elastically deformable material and has at least one pouch configured to receive a thermal unit for applying thermal therapy to a portion of the torso. Preferably, the garment 100 has at least two of such pouches. These pouches may include any or all of the following: an upper thoracic pouch 26, a thoracic pouch 28, a lumbar pouch 30, an abdominal pouch 32 and a breast pouch (not depicted). These pouches 26, 28, 30, 32 are substantially the same as depicted and described elsewhere herein with respect to other embodiments of the garment 100.

The curves of the spine 152 can be exaggerated in any plane, leading to pain, deformity and neurological dysfunction. All of the vertebral bodies act as a support column to hold up the spine and supports about half of the weight of the body, with the other half supported by the muscles. The areas of the body that are responsible for posture include the abdomen and the chest. By keeping the chest and abdomen in proper alignment, posture is optimized and many pains are alleviated or prevented.

The garment 100 provides support that redistributes weight to the portions of the body designed to carry weight. The shoulder straps 8, 10 allow a portion of the weight to be carried on the shoulders, relieving the spine of at least a portion of the weight. The overlapping design of the left abdominal flap 182 and right abdominal flap 184 around the abdomen and the attachment of the first end 16 and second end 18 in the thoracic area about the rear hip cooperate to redistribute weight over the hips and facilitates proper alignment of the spine. The attachment 84, 86 point between the midline 192 and the lateral 192 planes is an important feature. The attachment point 84, 86 will be a point of stress and weight bearing for the garment 100. If the attachment point 84, 86 were disposed along the back proper, that is the midline plane 192, weight and stress would be placed on the spine and would cause a pulling in the back-front direction. If the attachment point 84, 86 were disposed along the sides, that is along the lateral plane 190 (see FIG. 9), a pulling would be created in the side-to-side direction. By have two attachment points 84, 86 that are disposed between the lateral 190 and midline 192 (see FIG. 9) planes, there is no pulling force in either the forward-backward or side-to-side directions.

The garment 100 of the present invention functions as a support device, redistributing skeletal weight and pulling the spine into proper alignment. The garment supports by redistributing weight over the hips, shoulders and the back of rib cage where the skeleton is designed to carry weight. By way of illustration, when one bends forward repeatedly during the day, the weight tends to be pushed forward toward the abdomen. The garment pulls the spine into correct alignment, redistributing the body weight behind the pectoral muscles.

One feature of the supporting elements of the garment 100 includes the overlapping design of the left abdominal flap 182 and right abdominal flap 184 around the abdomen of the user. The left abdominal flap 182 and right abdominal flap 184 are in overlapping alignment when the garment 100 is worn by a user. This configuration provides double the abdominal support and bidirectional resistance. Additionally, as each abdominal flap 182, 184 connects to a hook or pile fastener 84, 86 in the area just behind the arm and over the corresponding rear hip, the support is evenly distributed about the torso and not shifted to either the front, back, right or left sides of the user. More specifically, a left abdominal flap 182 commences at the left side of a user and wraps around the front of the torso to attach to the right rear hip area. Correspondingly, the right abdominal flap 184 commences at the right side of a user and wraps around the front of the torso to attach to the left rear hip area. The two abdominal flaps 182, 184 are in overlapping alignment, but it is not important whether the right 184 or left 182 abdominal flaps are in the superior position.

In the maternity and female 200 embodiments, the bra element 46 additionally provide a supporting element. By redistributing weight to the shoulders via the shoulder straps 8, 10, the spine is not pulled forward by the breast weight.

FIGS. 4 and 5 depict a female configuration 200 of the garment. In some aspects, this is a maternity garment. As will be apparent, a maternity garment has a greater length at distance 170. This embodiment 200 is substantially the same as depicted and described with respect to embodiment 100 with the addition of a bra element 46. One embodiment of this bra element 46 for maternity and nursing is shown in greater detail on FIG. 11. Garment 200 in a maternity aspect may optionally also contain additional and/or larger abdominal pouches 32 that are advantageously used during post partum recovery. Optionally and additionally, breast pouches (not depicted) may be provided on the bra element 46. Thermal therapy may be used, for example, for relief from Cesarean sections and mammary discomfort from post partum engorgement, and/or mastitis.

Other prior art devices do not contemplate bra elements 46 with nursing features or other structures for exposing the breast tissues without removal of the device. In a maternity configuration of the garment 200 depicted in FIG. 11, the bra portion 46 comprises a nursing flap 57. Nursing bras are well known in the art and any suitable design may be incorporated. The nursing bra allows for a nursing flap 57 to be selectively detached to expose the breast and allow for infant nursing without removal of the garment 200. A breast pouch (not depicted) may also be located in the bra portion 46 similar to those previously depicted and described with respect to the upper thoracic pouch 26, thoracic pouch 28, lumbar pouch 30, and abdominal pouch 32.

Referring to FIGS. 4 and 11, the bra element 46 has a nursing flap 57 that comprises an outer flap 47 that reversibly attaches to an inner flap 49 by a mechanical fastener arrangement 50, 51. In the embodiment depicted, a hook or pile fastener 50 is provided along the periphery of the interior surface of the outer flap 47 and it's mating hook or pile fastener 51 is correspondingly located along the interior peripheral surface of the inner flap 49. The outer flap 47 and inner flap 49 form an inner cavity 53 where a nursing pad, thermal unit 40 or the like may be disposed during use. The outer-inner flap combination 47, 49 are reversibly attached to a base member 55 by two mechanical fasteners 44, 52, 48. The base member 55 is substantially triangular in shape or otherwise formed in the shape of a bra to surround and support the breast, having a cutout 45 in its central portion to allow an exposed breast. In the embodiment depicted, mechanical fastener 48 comprises a hook and eye arrangement. In the embodiment depicted, the second mechanical fastener arrangement comprises a hook 44 and a catch 52. As will be apparent, other mechanical fasteners may be suitably used to accomplish the intended function are considered within the scope of the present invention.

This nursing flap 57 feature may also be advantageously used in post operative applications where compression and thermal therapy in the breast region are desired by access to the breast is desired for regular dressing or drainage device changes, tissue and incision inspections, application of medicament creams, and the like. Optionally, a thermal unit 40 may be placed inside the bra portion 46 in addition to or in lieu of any breast pouch that may be provided.

In one aspect of this embodiment, a breast pouch is disposed on the exterior peripheral surface of each outer flap 47.

The foregoing description has been limited to specific embodiments of this invention. It will be apparent, however, that variations and modifications may be made by those skilled in the art to the disclosed embodiments of the invention, with the attainment of some or all of its advantages and without departing from the spirit and scope of the present invention. For example, the position or orientation and surface area of the hook or pile arrangements can be varied as desired. Several pieces of material may be sewn together to make a single garment. Different types of fabrics may be used in a single garment. The garment may have one or more openings for drainage tubes and the like, or connectors, clips, fasteners, and the like to hold or support other objects as needed.

It will be understood that various changes in the details, materials, and arrangements of the parts which have been described and illustrated above in order to explain the nature of this invention may be made by those skilled in the art without departing from the principle and scope of the invention as recited in the following claims.

I claim:

1. A garment wherein the garment
is configured to be worn in an annular manner by a user having a thoracic spine, a lumbar spine, a sacral spine, a cervical spine, and a torso;
is sized for positioning about the torso;
is disposed about at least a portion of the thoracic spine and at least a portion of the lumbar spine when worn by the user;
is formed from an elastically deformable material; and
comprises a plurality of pouches configured to receive a thermal unit for applying thermal therapy to a portion of the torso wherein the plurality of pouches comprises at least two pouches selected from the group consisting of an upper thoracic pouch, a thoracic pouch, a lumbar pouch, an abdominal pouch and a breast pouch.

2. The garment of claim 1 wherein the at least two thermal pouches comprises an upper thoracic pouch wherein the upper thoracic pouch delivers thermal therapy to at least a portion of the cervical spine and at least a portion of the thoracic spine.

3. The garment of claim 2 wherein
the thoracic spine comprises a plurality of thoracic vertebrae including thoracic vertebrae selected from the group consisting of T1, T2, T3, T4, T5, T6 and T7; and
the upper thoracic pouch is disposed about at least two thoracic vertebrae selected from the group consisting of T1, T2, T3, T4, T5, T6 and T7.

4. The garment of claim 1 wherein the at least two thermal pouches comprises a thoracic pouch wherein the thoracic pouch delivers thermal therapy to at least a portion the thoracic spine.

5. The garment of claim 4 wherein
the thoracic spine comprises a plurality of thoracic vertebrae including thoracic vertebrae selected from the group consisting of T9, T10, T11 and T12; and
the thoracic pouch is disposed about at least two thoracic vertebrae selected from the group consisting of T9, T10, T11 and T12.

6. The garment of claim 1 wherein the at least two thermal pouches comprises a lumbar pouch wherein the lumbar pouch delivers thermal therapy to at least a portion of the lumbar spine and at least a portion of the sacral spine.

7. The garment of claim 6 wherein
the lumbar spine comprises a plurality of lumbar vertebrae including lumbar vertebrae selected from the group consisting of L3, L4, and L5;
the sacral spine comprises a plurality of sacral vertebrae including sacral vertebrae selected from the group consisting of S1 and S2; and
the lumbar pouch is disposed about at least two vertebrae selected from the group consisting of L3, L4, L5, S1 and S2.

8. The garment of claim 1 wherein the garment is configured to simultaneously apply support, compression and thermal therapy to the torso.

9. The garment of claim 1 wherein the elastically deformable material comprises a material selected from the group consisting of a closed-cell neoprene, nylon, polyamide, polypropylene, polyester, TACTEL, SILKYLENE, LYCRA, SPANDEX, and combinations thereof.

10. The garment of claim 1 wherein the garment further comprises a bra element.

11. The garment of claim 10 wherein the bra element comprises a nursing flap.

12. The garment of claim 1 wherein the garment further comprises an auxiliary support strap.

13. The garment of claim 1 wherein the garment further comprises a thermal support strap.

14. The garment of claim 1 wherein the garment further comprises a hip cutout.

15. A garment for wearing in combination with an orthotic for the torso, wherein said garment:
is configured to be worn in an annular manner by a user having a thoracic spine, a lumbar spine, a cervical spine, a sacral spine, and a torso;
is sized for positioning about the torso;
is disposed about at least a portion of the thoracic spine and at least a portion of the lumbar spine when worn by the user;
is formed from an elastically deformable material; and
comprises at least one pouch configured to receive a thermal unit for applying thermal therapy to a portion of the torso.

16. The garment of claim 15 wherein the garment comprises at least two pouches configured to receive a thermal unit for applying thermal therapy to a portion of the torso selected from the group consisting of an upper thoracic pouch, a thoracic pouch, a lumbar pouch, an abdominal pouch and a breast pouch.

17. The garment of claim 16 wherein the upper thoracic pouch delivers thermal therapy to at least a portion the cervical spine and at least a portion of the thoracic spine; the thoracic pouch delivers thermal therapy to at least a portion the thoracic spine; and the lumbar pouch delivers thermal therapy to at least a portion of the lumbar spine and at least a portion of the sacral spine.

18. A garment for providing support, wherein said garment:
is configured to be worn in an annular manner by a user having a right rear hip, a left rear hip, an abdomen, a thoracic spine, a lumbar spine, and a torso;
is sized for positioning about the torso;
is disposed about at least a portion of the thoracic spine and at least a portion of the lumbar spine when worn by the user;
is formed from an elastically deformable material;
comprises a hip cutout;
comprises a left abdominal flap and a right abdominal flap that are disposed in overlapping alignment about the abdomen when the garment is worn by the user;
comprises two front shoulder straps and two back shoulder straps that cooperate to apply compression vertically across the torso;
comprises a first connecting section disposed about the left rear hip, wherein the first connecting section and the right abdominal flap cooperate to apply compression horizontally across the torso; and
comprises a second connecting section disposed about the right rear hip, wherein the second connecting section and the left abdominal flap cooperate to apply compression horizontally across the torso.

19. The garment of claim 18 wherein the garment further comprises at least one pouch configured to receive a thermal unit for applying thermal therapy to a portion of the torso.

20. The garment of claim 18 wherein the garment further comprises an auxiliary support strap formed from an elastically deformable material and configured to be worn on the torso in an annular fashion and apply compression horizontally across the torso.
